(19) United States
(12) Patent Application Publication
(10) Pub. No.: US 2020/0325429 A1
(43) Pub. Date: Oct. 15, 2020

(54) CELL CULTURE DEVICE

(71) Applicant: Panasonic Corporation, Osaka (JP)

(72) Inventors: Takeshi ANDO, Kyoto (JP); Toshinori HIROSE, Tokyo (JP); Norihiro SHIBATA, Osaka (JP); Hisato NAKAJIMA, Osaka (JP); Toshiaki YAMAUCHI, Kyoto (JP)

(21) Appl. No.: 16/913,148

(22) Filed: Jun. 26, 2020

Related U.S. Application Data
(63) Continuation of application No. 16/061,984, filed on Jun. 13, 2018.

(30) Foreign Application Priority Data
Feb. 3, 2016 (JP) 2016-018818

(51) Int. Cl. C12M 3/00; C12M 1/00; G06K 9/32; G06K 9/20; C12M 1/34; C12M 1/32; G06K 9/00

(52) U.S. Cl. CPC C12M 3/00; C12M 23/50; C12M 41/14; G06K 9/325; G06K 9/2054; G06T 7/0012; C12M 23/12; C12M 23/34; G06K 9/00134; G06K 2209/015; C12M 41/36

(57) ABSTRACT

Cell culture device includes incubator unit that cultures cells in culture container, transport unit that transports culture container to incubator unit, task setting unit that sets a task relating to a culture of the cells, imaging unit serving as an example of a management information acquisition unit that acquires management information attached to culture container, and storage unit that stores the management information acquired by imaging unit. From storage unit, task setting unit reads the management information of plurality of culture containers serving as targets for carrying out the task, and causes display serving as an example of a display unit to display the management information.

### CROSS-REFERENCE TO RELATED APPLICATIONS

This application is a U.S. national stage application of the PCT International Application No. PCT/JP2017/002247 filed on Jan. 24, 2017, which claims the benefit of foreign priority of Japanese patent application 2016-018818 filed on Feb. 3, 2016, the contents all of which are incorporated herein by reference.

### TECHNICAL FIELD

The present disclosure relates to a cell culture device which automatically cultures cells.

### BACKGROUND ART

A cell culture device which automatically culture cells is used in order to stabilize quality of the cells by culturing a large amount of the cells in a regenerative medical field or in a drug discovery field. For example, cultured cells are returned to a human body in the regenerative medical field, and are used for testing drug efficacy and side effects in the drug discovery field. Accordingly, the quality of the cells cultured using the cell culture device is very important.

For example, as a technique for managing the quality of the cells, PTL 1 discloses the following technique. A growth state of the cells adhering to a bottom surface of a culture container and an adhesion state between the bottom surface of the culture container and the cells are observed using a microscope for each culture process. In this manner, quality management is performed on the cells. Furthermore, work management is performed on culturing operations such as culture medium exchange or successive subculture performed in the culture process.

In recent years, as a culture technique, a chemical solution (culture solution), and a robot technique have been developed, types of the cells which can be cultured using cell culture device have increased. However, the whole culture process of the cells has not yet been automated. For example, in a case where an induced pluripotent stem cell (iPS cell) is cultured, it is difficult to automate an introduction process of introducing a plurality of genes into somatic cells or the subsequent initial process. Consequently, manual work is required. On the other hand, processes after the above-described processes (for example, a culture process for maintaining an undifferentiated state of the iPS cells) are automated. In this case, a manual culture for manually carrying out culture work is changed to an automatic culture for carrying out the culture work by using the cell culture device.

### CITATION LIST

### Patent Literature

PTL 1: International Publication No. WO2012/127879

### SUMMARY

### Technical Problem

However, according to the cell culture device in the related art including the technique disclosed in PTL 1, consideration is given to the quality management or the work management of cells within an automated process. However, no sufficient consideration is given to the quality management or the work management of cells when the manual culture is changed to the automatic culture. As a result, when the manual culture is changed to the automatic culture, there is a possibility that management information used during the manual culture may be mistakenly handed over to the automatic culture.

An object of the present disclosure is to solve the above-described problem by provide a cell culture device capable of accurately handing over management information used during a manual culture to an automatic culture, when the manual culture is changed to the automatic culture.

### Solutions to Problem

According to an aspect of the present disclosure, there is provided a cell culture device including an incubator unit, a transport unit, a task setting unit, a management information acquisition unit, and a storage unit. The incubator unit cultures cells in a culture container. The transport unit transports the culture container to the incubator unit. The task setting unit sets a task relating to the culturing of the cells. The management information acquisition unit acquires management information attached to the culture container. The storage unit stores the management information acquired by the management information acquisition unit. From the storage unit, the task setting unit reads the management information of the culture container selected from the plurality of culture containers serving as a target for carrying out the task, and causes a display unit to display the management information.

### Advantageous Effect of Invention

According to the cell culture device in the aspect of the present disclosure, when the manual culture is changed to the automatic culture, it is possible to accurately hand over the management information used during the manual culture to the automatic culture.

### BRIEF DESCRIPTION OF DRAWINGS

FIG. 1 is a view illustrating a configuration example of a cell culture device according to an embodiment.

FIG. 2 is a view illustrating an example of management information attached to an upper surface of a culture container according to the embodiment.

FIG. 3 is a view illustrating an example of a storage state of the culture container inside an incubator unit according to the embodiment.

FIG. 4 is a view illustrating an example of a container selection screen displayed on a display according to the embodiment.

FIG. 5 is a flowchart illustrating an operation example in the cell culture device according to the embodiment.

### DESCRIPTION OF EMBODIMENT

Hereinafter, an embodiment will be described with reference to the drawings. The same reference numerals will be given to the same configuration elements in each drawing, and description thereof will be omitted in some cases. In order to facilitate understanding, in each drawing, each configuration element is schematically illustrated as a main constituent.

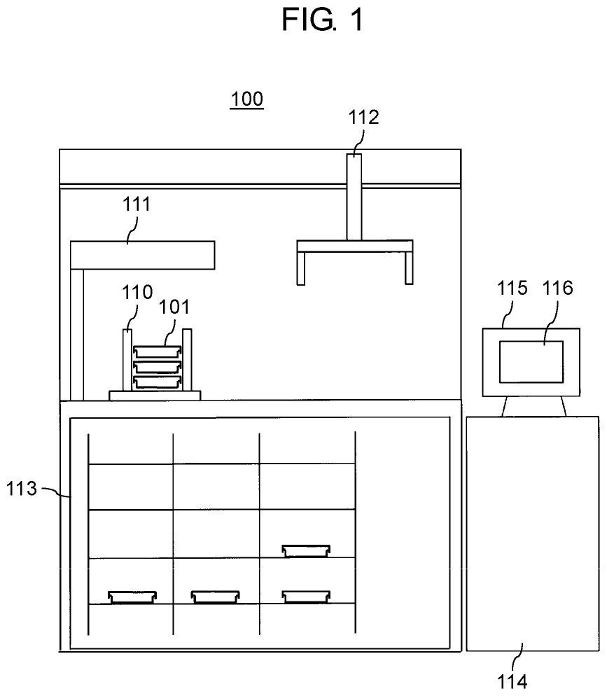

First, referring to FIG. 1, a configuration of cell culture device 100 according to the embodiment will be described. FIG. 1 is a view illustrating a configuration example of cell culture device 100 according to an embodiment.

As illustrated in FIG. 1, cell culture device 100 includes loading unit 110, imaging unit 111, transport unit 112, incubator unit 113, storage unit 114, and task setting unit 115. Hereinafter, each unit will be described.

Loading unit 110 is a place where culture container 101 is located.

Here, culture container 101 is a plastic container such as a dish and a well plate, for example. Cells are cultured in culture container 101. Culture container 101 is manually located in loading unit 110.

Imaging unit 111 (example of a management information acquisition unit) images an appearance of culture container 101 located in the loading unit 110. As an imaging timing, the appearance is imaged before transport unit 112 transports culture container 101 located in loading unit 110 to incubator unit 113.

Management information of the cells cultured in culture container 101 is indicated on the appearance of imaged culture container 101.

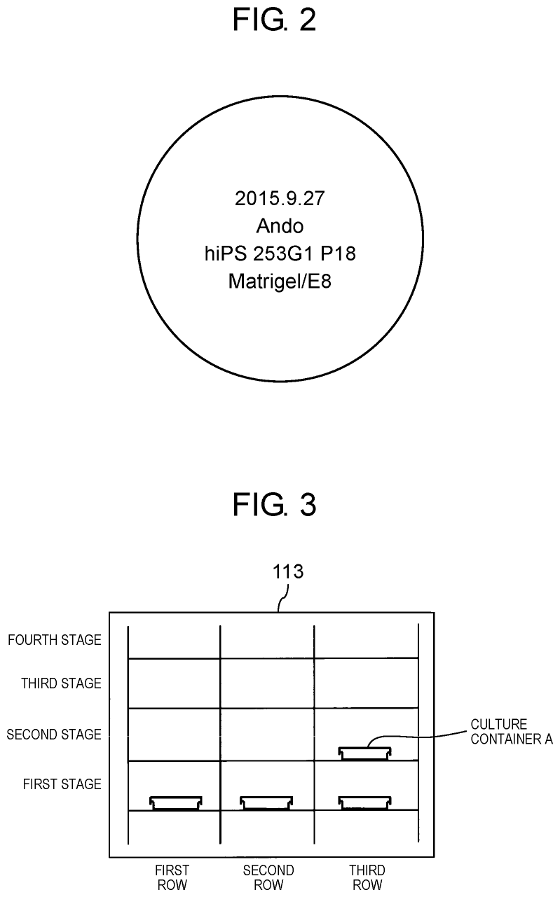

Here, referring to FIG. 2, the management information will be described. FIG. 2 is a view illustrating an example of the management information attached to an upper surface of culture container 101 (refer to FIG. 1) according to the embodiment.

As illustrated in FIG. 2, in a case where culture work is manually carried out, the management information for management targets in a culture process is indicated using handwriting on a lid portion of the upper surface of culture container 101 (refer to FIG. 1). As illustrated in FIG. 2, for example, the management information includes information on a date when culture starts (“Sep. 27, 2015” illustrated in FIG. 2), information on a culture person (“Ando” illustrated in FIG. 2), information on a type of cells (“hiPS” illustrated in FIG. 2), information on a name of a cell line (“253G1” illustrated in FIG. 2), information on the number of successive subcultures of cells (“P18” illustrated in FIG. 2), information on a type of coating agents of culture container 101 (“Matrigel” illustrated in FIG. 2), and information on a type of culture media (“E 8” illustrated in FIG. 2). Information included in the management information is not limited to the information illustrated in FIG. 2.

Hitherto, the management information has been described. Hereinafter, referring back to FIG. 1, description will be continued.

For example, imaging unit 111 images the appearance of the upper surface of culture container 101 illustrated in FIG. 2, and stores an image (picture) in storage unit 114.

Transport unit 112 transports culture container 101 located in loading unit 110 to imaging unit 111, and after the appearance is imaged, transport unit 112 transports culture container 101 from imaging unit 111 to incubator unit 113. Transport unit 112 transports culture container 101 from incubator unit 113 to a work unit (not illustrated) in which a culture solution contained in culture container 101 is aspirated and discharged. Transport unit 112 transports a chemical solution from a refrigerator (not illustrated) to a heater unit (not illustrated) in a state where a pipette tip is attached. Transport unit 112 transports the chemical solution from the heater unit to the work unit. Transport unit 112 transports the chemical solution from the work unit to a waste liquid unit (not illustrated).

In this way, transport unit 112 transports culture container 101, the chemical solution, the pipette tip, and a tube for storing the chemical solution.

Incubator unit 113 cultures the cells in culture container 101 under a predetermined environment. Inside incubator unit 113, culture container 101 transported by transport unit 112 is stored in the predetermined environment (for example, CO₂ concentration is 5% and temperature is 37° C.). A storage position of culture container 101 inside incubator unit 113 (where is a position of culture container 101 stored inside incubator unit 113) is managed by cell culture device 100 as follows.

Referring to FIG. 3, management of the storage position of culture container 101 inside incubator unit 113 will be described. FIG. 3 is a view illustrating an example of the storage position of culture container 101 inside incubator unit 113 according to the embodiment.

For example, as illustrated in FIG. 3, a plurality of the storage positions divided into cellular shapes are present inside incubator unit 113. Culture containers 101 (refer to FIG. 1) are stored one by one at each of the storage positions. For example, regarding culture container A illustrated in FIG. 3, storage position information indicating that the storage position of culture container A is located in a second stage of a third row is managed. The storage position information is stored in storage unit 114 (refer to FIG. 1).

In storage unit 114, an appearance image (image of the management information) of culture container 101 imaged by imaging unit 111 (refer to FIG. 1) is associated with the storage position information. The storage position information includes loading date and time information indicating a date and time when culture container 101 is loaded, operation information relating to a mechanical operation of cell culture device 100 (for example, information indicating a discharge amount and discharge speed of the chemical solution), task information indicating completed or scheduled tasks (details will be described later), a cell state image obtained by imaging a growth state of the cells during the culture, environmental information indicating an environment inside cell culture device 100 (for example, information indicating temperature and humidity and a degree of cleanness), and culture method information indicating a method of culturing the cells. These pieces of information together with various pieces of information (refer to FIG. 2) included in the appearance image of culture container 101 can be called the management information. These pieces of information are appropriately read from storage unit 114 by task setting unit 115 (refer to FIG. 1), and are displayed on a container selection screen (to be described later).

The storage position information described above is used for a transport operation of culture container 101 which is performed by transport unit 112 (refer to FIG. 1). For example, in a case where transport unit 112 picks up culture container 101 having a task set therein from incubator unit 113 and thereafter transport unit 112 transports culture container 101 to incubator unit 113 again, culture container 101 is located at the storage position indicated by the storage position information. In this manner, culture container 101 is stored at the same storage position.

Storage unit 114 stores the appearance image of culture container 101 imaged by imaging unit 111, storage location information, and information other than the management information included in the appearance image (for example, the above-described loading date and time information or task information) in association with each other.

Task setting unit 115 sets scheduled tasks. For example, the tasks include culture medium exchange work, successive subculture work, observation work, and unloading work.

For example, task setting unit 115 causes display 116 (example of a display unit, refer to FIG. 1) to display a task selection screen (not illustrated) indicating a list of selectable tasks. In an example illustrated FIG. 1, a configuration is adopted so that display 116 is provided in task setting unit 115. However, display 116 may be provided outside task setting unit 115 or outside cell culture device 100.

If a user selects a desired task on the task selection screen, task setting unit 115 reads the above-described various pieces of information from storage unit 114, and causes display unit 116 to display a container selection screen including the read information.

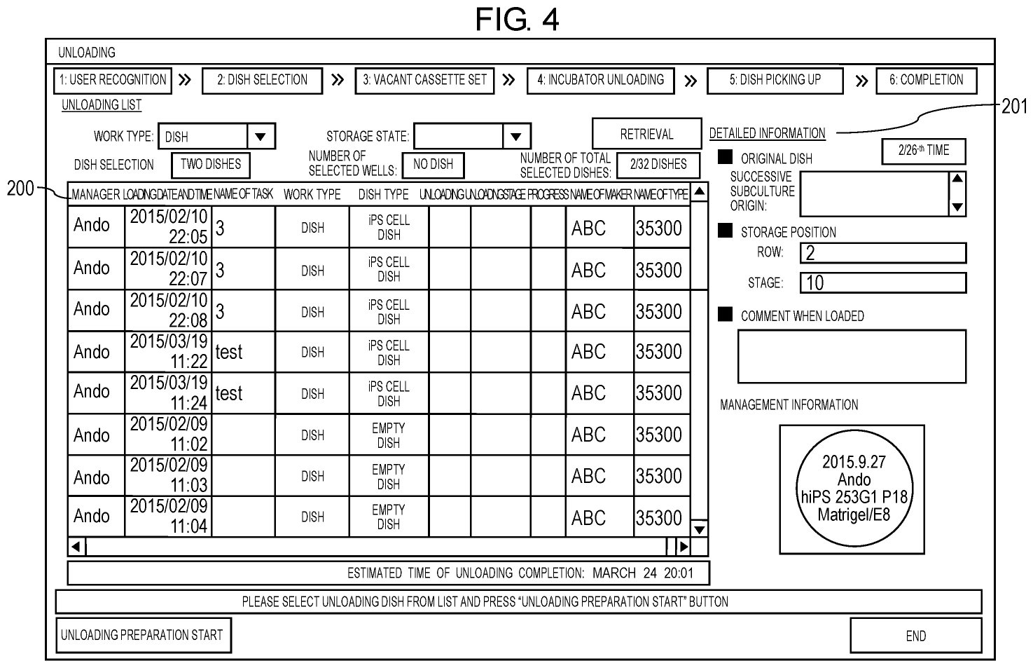

Here, referring to FIG. 4, an example of the container selection screen will be described. FIG. 4 is a view illustrating an example of the container selection screen displayed on display 116 (refer to FIG. 1) according to the embodiment. For example, the container selection screen illustrated in

FIG. 4 is displayed when unloading work is selected by the user. However, in a case where other tasks (for example, the culture medium exchange work, the successive subculture work, and the observation work) are selected, the same container selection screen is also displayed.

As illustrated in FIG. 4, container list information 200 and detailed information 201 are displayed on the container selection screen. Container information 200 includes information such as an manager, the loading date and time, a name of the completed task, and a type of culture containers which are stored for each culture container 101 (those which are stored in incubator unit 113 (refer to FIG. 1), refer to FIG. 1) serving as targets of the unloading work. Detailed information 201 includes information such as the storage position of culture container 101 in incubator unit 113, a comment input by a user during the loading, and the appearance image (FIG. 4 illustrates the “management information”) of culture container 101 imaged by imaging unit 111 (refer to FIG. 1).

For example, if the user performs an operation of selecting desired culture container 101 from culture containers 101 included in container list information 200, detailed information 201 (storage position, comment, and appearance image) relating to selected culture container 101 is displayed. Detailed information 201 is not limited to the example illustrated in FIG. 4.

When the user selects culture container 101, the user can confirm the management information relating to culture cells of culture container 101 by using the appearance image of culture container 101. Therefore, the user can reduce errors in selecting culture container 101 when the user performs task setting.

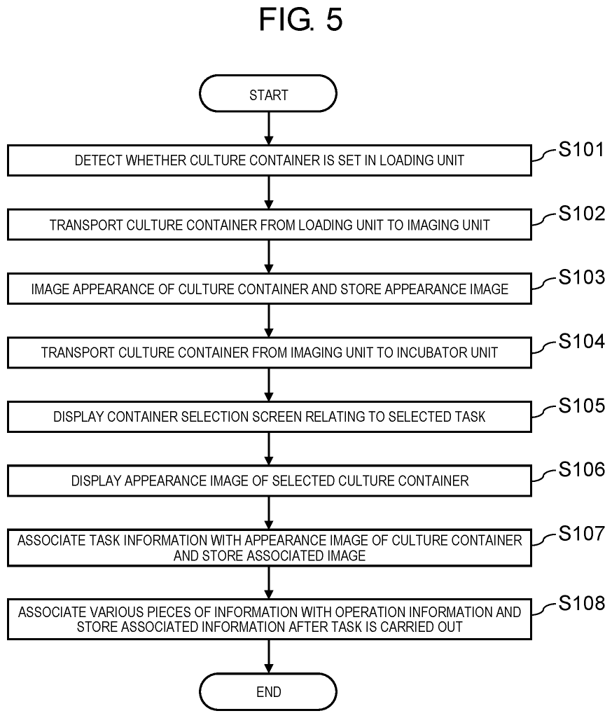

Next, referring to FIG. 5, an operation example of cell culture device 100 according to the present embodiment will be described. FIG. 5 is a view illustrating the operation example of cell culture device 100 according to the embodiment. Specifically, FIG. 5 is a view illustrating a process in which culture container 101 is loaded in cell culture device 100 and a task is carried out for culture container 101.

First, loading unit 110 detects whether culture container 101 is set in loading unit 110 (Step S101).

Next, transport unit 112 transports set culture container 101 from loading unit 110 to imaging unit 111 (Step S102).

Next, imaging unit 111 images the appearance of culture container 101 (portion having the management information attached thereto), and stores the captured appearance image in storage unit 114 (Step S103).

Next, transport unit 112 transports culture container 101 from imaging unit 111 to a predetermined storage position of incubator unit 113 (Step S104).

Next, task setting unit 115 causes display 116 to display the container selection screen relating to the selected task (Step S105). As described above, the task herein is selected on the task selection screen displayed on display 116 before the container selection screen is displayed.

Next, task setting unit 115 causes display 116 to display the appearance image of selected culture container 101 (Step S106).

This appearance image includes the management information. In addition to the appearance image, the storage position or the comment of culture container 101 is displayed on display 116. With reference to these pieces of information displayed on display 116, a user determines culture container 101 for carrying out the task.

Next, task setting unit 115 associates task information indicating the selected scheduled task with the appearance image of selected culture container 101, storage position information of selected culture container 101, task information indicating the completed task in selected culture container 101, and causes storage unit 114 to store the associated information (Step S107).

After the selected task is carried out, task setting unit 115 associates various pieces of information (for example, the appearance image, the storage position information, and the task information) which are associated with each other in Step S107 with operation information (information indicating the discharge amount or the discharge speed of the chemical solution) relating to an operation performed by a mechanical element (for example, a chemical solution suction pump) disposed inside cell culture device 100 when the task is carried out, and causes storage unit 114 to store the associated information (Step S108). The associated information herein is not limited to the operation information, and may include the loading date and time information, the environment information, the cell state image, and the culture method information which are described above.

As described above, cell culture device 100 according to the present embodiment includes incubator unit 113, transport unit 112, task setting unit 115, imaging unit 111 serving as the management information acquisition unit, and storage unit 114. Incubator unit 113 cultures the cells in culture container 101. Transport unit 112 transports culture container 101 to incubator unit 113. Task setting unit 115 sets the task relating to the cell culture. Imaging unit 111 serving as the management information acquisition unit acquires the management information attached to culture container 101. Storage unit 114 stores the management information acquired by imaging unit 111 serving as the management information acquisition unit. From storage unit 114, task setting unit 115 reads the management information of culture container 101 selected from plurality of culture containers 101 serving as targets for carrying out the task, and causes display 116 to display the management information.

In this manner, after the user selects a desired task, when the user selects culture container 101 for carrying out the task, the management information of culture container 101 is displayed on display 116. The user can select culture container 101 with reference to the management information. Accordingly, the user can reduce errors in selecting culture container 101. Therefore, when a manual culture is changed to an automatic culture, the management information used during the manual culture can be accurately handed over to the automatic culture.

According to cell culture device 100 of the present embodiment, imaging unit 111 images the management information attached to culture container 101, storage unit 114 stores the image obtained by imaging the management information, and task setting unit 115 causes display 116 to display the image.

In this manner, the user can more visibly confirm the management information attached to culture container 101 by using the image. Therefore, the user can more easily select culture container 101 serving as a target.

Hitherto, the embodiment according to the present invention has been described. However, the present invention is not limited to the above-described embodiment. Various modifications can be made within the scope not departing from the gist of the present invention.

For example, in the embodiment, a case where the appearance image of culture container 101 imaged by imaging unit 111 is stored has been described as an example. However, the present invention is not limited thereto. For example, a character recognition unit (not illustrated, an example of the management information acquisition unit) may perform a character recognition process on the appearance image. In this manner, the management information (for example, character information indicating the culture person, the date, the cell line, the number of successive subcultures, the coating material, the culture medium, and the additive) may be extracted, and the management information may be stored in storage unit 114 in association with the appearance image. In this manner, it is possible to increase the amount of information relating to the task in Step S108, and it is possible to improve traceability of the cells cultured by the cell culture device after the task is carried out. For example, as the timing for the character recognition process, the character recognition process is performed before transport unit 112 transports culture container 101 located in loading unit 110 to incubator unit 113.

In imaging unit 111, in a case where culture container 101 is provided with a transparent lid, character recognition may be performed from a region where 50% of an area of the lid is excluded from a central portion. In this manner, in the cell culture, depending on a type of the cells or a culture state, handwritten information may not be distinguished from the cells and may be less likely to be identified in some cases. However, if a recognizable range is limited, information can be stably identified in the cell culture.

The management information (for example, at least one of the respective pieces of information illustrated in FIG. 2) extracted by performing the character recognition may be used as a retrieval condition of culture container 101 displayed on the container selection screen. For example, in a case where culture start date and time which are subjected to the character recognition are used as the retrieval condition, it is possible to selectively determine culture container 101 corresponding to the management information including the information on the culture start date and time, and selectively determined culture container 101 can be displayed on the container selection screen. In this manner, a user can further reduce errors in selecting culture container 101.

For example, in the embodiment, a case where the management information is indicated by handwriting on the lid portion on the upper surface of culture container 101 has been described as an example. However, an identification tag including the management information, for example, a barcode may be attached to the lid portion on the upper surface of culture container 101. In this case, cell culture device 100 causes a barcode reader (not illustrated, an example of the management information acquisition unit) to read the barcode. In this manner, cell culture device 100 extracts the management information (for example, character information indicating the culture person, the date, the cell line, the number of successive subcultures, the coating material, the culture medium, and the additive), and stores the management information in the storage unit 114. For example, as the timing for reading the barcode, the barcode is read before transport unit 112 transports culture container 101 located in loading unit 110 to incubator unit 113. In this way, instead of the appearance image, the management information is displayed using text on the container selection screen. In this manner, cell culture device 100 can more accurately acquire the management information. The management information included in the barcode is manually input, for example.

For example, as the identification tag, in addition to the barcode, a QR code (registered trademark) or an integrated circuit (IC) chip can be used. As the management information acquisition unit corresponding to the identification tags, a QR code reader or an IC reader can be used.

A user using cell culture device 100 may be specified from the management information recognized by imaging unit 111. When culture container 101 is stored in incubator unit 113, culture container 101 may be stored for each user. In this manner, it is possible to know the amount of culture container 101 used for each user, and it is possible to reduce the risk that culture container 101 may be used by another user.

When culture container 101 is loaded to cell culture device 100, a moving image obtained by recording the culture process up to the loading process may be stored in association with the management information. In this manner, much more information can be associated with culture container 101, and the traceability can be improved.

### INDUSTRIAL APPLICABILITY

The cell culture device according to the aspect of the present disclosure is useful in a field requiring treatment or examination using the cells such as the regenerative medical field and the drug discovery field.

### REFERENCE MARKS IN THE DRAWINGS

100 CELL CULTURE DEVICE
101, A CULTURE CONTAINER
110 LOADING UNIT
111 IMAGING UNIT (MANAGEMENT INFORMATION ACQUISITION UNIT)
112 TRANSPORT UNIT
113 INCUBATOR UNIT
114 STORAGE UNIT
115 TASK SETTING UNIT
116 DISPLAY (DISPLAY UNIT)

1-8. (canceled)

9. A method for culturing cells in a culture container, the culture container being one of a plurality of culture containers, the method comprising:
creating an image of an appearance of the culture container, the appearance of the culture container including management information of the cells in the culture container;
storing the image of the appearance of the culture container;
transporting the culture container to an incubator;
culturing the cells in the culture container by the incubator;
displaying, on a display, a list of selectable tasks related to the culturing of cells;
selecting one of the tasks on the display;
displaying, on the display, a culture container selection screen relating to the selected one of the tasks;
selecting the culture container from the culture container selection screen; and
displaying, on the display, the stored image of the selected culture container.

10. The method of claim 9,
wherein the selecting of the culture container includes selectively determining the culture container through character recognition of the management information included in the image of the culture container.

11. The method of claim 9, further comprising:
extracting character information by performing a character recognition process on the management information included in the image of the culture container; and
storing the character information in association with the image.

12. The method of claim 11,
wherein the culture container is provided with a transparent lid, and the character recognition process is performed on a region where 50% of an area of the transparent lid is excluded from a central portion in the image.

13. The method of claim 11,
wherein the culture container is provided with an identification tag having the management information, and
wherein the character recognition process is performed on the identification tag.

14. The method of claim 9,
wherein the management information includes a specified user who is associated with the culture container,
the method further comprising storing the culture container in the incubator for the specified user.

15. The method of claim 9, further comprising:
creating a moving image by recording a culture process performed before the transporting of the culture container to the incubator; and
storing the moving image in association with the image including the management information.

16. The method of claim 9, wherein the creating of the image of the appearance of the culture container is performed before the transporting of the culture container to the incubator.

17. The method of claim 9, further comprising:
writing the management information on a lid portion of the culture container.
